(19) United States
(12) Patent Application Publication
(10) Pub. No.: US 2020/0063109 A1
(43) Pub. Date: Feb. 27, 2020

(54) PHAGE COMPOSITION AND USE THEREOF IN INACTIVING ANTIBIOTIC RESISTANCE PATHOGENIC BACTERIA

(71) Applicant: Institute of Social Science, Chinese Academy of Sciences, Nanjing (CN)

(72) Inventors: Mao YE, Nanjing (CN); Yuanchao ZHAO, Nanjing (CN); Mingming SUN, Nanjing (CN); Zhongyun ZHANG, Nanjing (CN); Dan HUANG, Nanjing (CN); Xin JIANG, Nanjing (CN)

(73) Assignee: Institute of Social Science, Chinese Academy of Sciences, Nanjing (CN)

(21) Appl. No.: 16/547,059

(22) Filed: Aug. 21, 2019

(30) Foreign Application Priority Data
Aug. 22, 2018 (CN) 201810959955.8

(51) Int. Cl. C12N 7/00; B09C 1/10

(52) U.S. Cl. CPC C12N 7/00; C12N 2795/10321; B09C 1/10

(57) ABSTRACT

A phage composition and use thereof in inactivating antibiotic resistance pathogenic bacteria are disclosed. The phage composition includes three phages, and the phages all have been deposited at the China Center for Type Culture Collection on Aug. 1, 2018. The phages are a phage φYSZKA under Accession No. CCTCC M 2018513, a phage φYSZKP under Accession No. CCTCC M 2018514, and a phage φYSZPA under Accession No. CCTCC M 2018515, respectively. The present invention provides a remediation method by applying a specific bacteriophage stock solution into a contaminated soil-plant system to directionally infect and inactivate a combined contamination of resistance pathogenic bacteria in the system and synergistically remove antibiotic resistance genes. In addition, the functional diversity and stability of a soil ecological environment are significantly restored, after the remediation.

### FIELD OF THE INVENTION

The present invention belongs to the technical field of remediation of compound contaminated soil of multiple antibiotic resistance pathogenic bacteria, and particularly relates to a method for targetedly inactivating antibiotic resistance pathogenic bacteria in a soil-vegetable system by polyvalent phage therapy.

### DESCRIPTION OF RELATED ART

Bacteriophages (abbreviated as phages) are a kind of organisms surviving by exclusively preying on living host bacteria, and are widely distributed in soil, water, air, and even human and animal body surfaces or intestinal tracts. It is estimated that the total amount of the phages reaches 10³¹ population. The polyvalent phage therapy refers to a remediation method of separating, screening, purifying and enriching exclusive phages of host bacteria, then artificially accelerating the expression of a broad host spectrum, screening polyvalent phages with high titer, short lysis period and strong stress resistance, and then adding a specific bacteriophage stock solution to a contaminated soil-plant system to directionally infect and inactivate pathogenic bacteria.

Furthermore, in recent years, due to the abuse of antibiotic veterinary drugs, the defect of a safe treatment technology for livestock and poultry manure, and the lack of environmental management, in China and many countries around the world, farmland soil-vegetable systems around suburban livestock farming plants often become high-risk hotspot “sources” and “sinks” for residue and breeding of antibiotic resistance bacteria (ARB) and antibiotic resistance genes (ARGs). Especially, under the promotion of horizontal transfer or vertical transduction of a large number of movable gene elements (plasmids, integrons, and transposons) in an environment, the risk of spread of some zoonotic antibiotic resistance pathogenic bacteria is greatly increased, and furthermore, it causes a very serious potential threat to human health and ecological safety. Therefore, it is very necessary and urgent to develop the technical invention of the polyvalent phage therapy for targetedly inactivating antibiotic resistance pathogenic bacteria in a soil-vegetable system.

Through relevant literature review and patent search, publication and acceptance of the polyvalent phage therapy for the biological remediation technology for a compound high-abundance antibiotic resistance pathogenic bacterium and resistance gene contaminated soil-vegetable system are not found. The existing methods most similar to the present invention are phage therapy for mammalian pathogenic bacterium infection and phage therapy related to bacterial wilt of crops. In patent applications CN201580060307.3, CN201580008049.4, CN201610924016.0, CN201610014708.1, and CN201510008569.7, high-specificity phages attack and inactivate Pseudomonas aeruginosa, colon bacillus, Xanthomonas campestris, and Ralstonia solanacearum respectively. At present, the existing phage therapy is mainly an inactivation process by exclusively attacking on a host bacterium, but the remediation technologies for the compound contamination of resistance pathogenic bacteria in a soil-vegetable system are very rare. The patent applications CN201580008049.3 and CN201580008049.4 respectively provide a method for treating Pseudomonas aeruginosa/colon bacillus infection by a phage combined agent, and the present invention uses specific pathogenic bacteria as host bacteria to screen a large amount of phages in environmental samples, and selects a high-activity phage combination as a raw material for preparation of an agent, and “many-to-one” efficient inactivation can be performed for Pseudomonas aeruginosa/colon bacillus. The patent application CN201610924016.0 provides a technology for applying salmonella phage and a mixture thereof in a food system, and the method mainly screens the exclusive virulent phages of Salmonella enteritidis and Salmonella typhimurium from a soil environment, and uses a mixture of the two phages to control the spread of salmonella bacteria in the food safety field. The patent application CN201610014708.1 provides a biological control technology for rice leaf blight by phage therapy, and the method mainly screens a virulent phage for inactivating Xanthomonas campestris of rice leaf blight from the soil environment with high specificity, and aims at prevention and treatment of rice leaf blight. The patent application CN201510008569.7 provides a technical method for controlling tobacco bacterial wilt by phage therapy, and the method mainly uses a sterile injector to inject prepared phage suspension to tobacco stems and uses mineral oil to cover the outer side. The four methods apply phages to pathogenic sites for specific attack and inactivation of pathogenic bacteria, which lack an overall remediation effect on synergistic removal of pathogenic bacteria from a soil-crop (vegetable) system. Furthermore, the existing patents do not involve the introduction for inactivation of pathogenic bacteria carrying antibiotic resistance genes in a soil-vegetable system by use of phage therapy.

The prior art has the following main defects: most of the existing phage therapies select exclusive high-specificity phages as raw materials, one phage correspondingly “preys on” one host bacterium, the treatment system is too single, the preparation of the agent is complicated, a biological control technology for simultaneous inactivation of the compound contamination of multiple antibiotic resistance pathogenic bacteria in a soil-vegetable system is lacking, and furthermore, the related ecological risk assessment of the functional stability and diversity of microflora in a soil-vegetable system after application has received little attention.

The main reasons for the defects are as follows: In recent years, the academic community has gradually recognized that a soil-vegetable system is a “source” and “sink” for accumulation of multiple antibiotic resistance bacteria and resistance genes, and this type of novel resistance pathogenic bacteria and genes will seriously threaten the human health and ecological environment safety through the transfer action of a food chain. In most of the existing researches, one or more phages specifically attack a certain “species” of host bacteria, and less attention is paid to the contamination of high-abundance compound pathogenic bacteria in a soil-vegetable system. Therefore, it is urgent to carry out the research and development of a bio-targeted inactivation technology for specifically reducing and eliminating the risk of accumulation of antibiotic resistance pathogenic bacteria in a soil-vegetable system.

### SUMMARY OF THE INVENTION

### Technical Problem

Aiming at the defects of the prior art, the present invention provides a phage composition and application thereof in inactivating antibiotic resistance pathogenic bacteria. The method adopts a remediation mode of separating and purifying exclusive phages of host bacteria, then artificially accelerating the expression process of a broad host spectrum to obtain a polyvalent phage capable of simultaneously attacking different kinds of pathogenic bacteria, and adding a specific bacteriophage stock solution to a contaminated soil-plant system to directionally infect and inactivate a combined contamination of resistance pathogenic bacteria in the system and synergistically remove antibiotic resistance genes. After the remediation, the functional diversity and stability of a soil ecological environment are significantly restored. The method is a biological remediation technology with environmental friendliness.

### Technical Solution

A phage composition includes three phages, the phages all have been deposited at the China Center for Type Culture Collection on Aug. 1, 2018, and the phages are a phage φYSZKA under Accession No. CCTCC M 2018513 and the classification name is Klebsiella phage φYSZKA, a phage φYSZKP under Accession No. CCTCC M 2018514 and the classification name is Klebsiella and Pseudomonas aeruginosa phage φYSZKP, and a phage φYSZPA under Accession No. CCTCC M 2018515 and the classification name is Pseudomonas aeruginosa phage φYSZPA, respectively. They are deposited at China Center for Type Culture Collection, Wuhan University, Luojia Hills, Wuchang, Wuhan, Hubei Province.

The phage composition is used for targetedly inactivating antibiotic resistance pathogenic bacteria in a soil-vegetable system.

The phage composition is used for preparing a product for targetedly inactivating antibiotic resistance pathogenic bacteria in a soil-vegetable system.

The working principle of the present invention is as follows: 1. phages are a kind of bacterial viruses consisting of protein capsids (60%) and nucleic acids (40%) and having no intact mature cell structure, can survive by specifically “preying on” host bacteria, and can be divided into lytic phages and lysogenic phages; 2. virulent phages can recognize specific binding sites of the surfaces of cell membranes of host bacteria in an environmental migration process and perform paired adsorption, subsequently, the tail sheaths of the phages shrink, the DNA of the nucleic acids is injected into the host bacteria through hollow tails to execute an invasion process, then, the DNA of the phages utilizes nucleic acid base pairs and energy substances in the host to rapidly complete its own nucleic acid replication and protein synthesis, a large number of progeny phages are assembled and proliferated in the bacteria, and cell wall lytic enzymes are released, thereby causing the host bacteria to rupture and die and destroying the internal structure of the bacteria so as to finally complete lysis and release processes; 3. a polyvalent phage refers to a phage capable of attacking two or more homology-similar “species” of different species of host bacteria; 4. a polyvalent phage with high titer, short lysis period and strong stress resistance is selected by simulation according to in-situ contaminated soil environmental conditions (temperature, pH, ion concentration, and the like) so as to be used as a preferred strain for polyvalent phage therapy, the polyvalent phage will preferentially “prey on” pathogenic bacteria with higher specificity in an environment, after this type of pathogenic bacteria are reduced to a certain level, the polyvalent phage will attack host bacteria with weaker specificity and maintain its survival state at a certain level, and furthermore, secondary “rebound” of the pathogenic bacteria can be prevented; 5. the length of the phage is about 20-200 μm, which is equivalent to one divided by several hundreds of that of bacteria or one thousandth of that of bacteria, and in the soil-vegetable system, some phages will be transferred into vegetables by means of plant root penetration and leaf surface transpiration to synchronously track and inactivate resistance pathogenic bacteria in the vegetables, thereby preventing the spread of the resistance pathogenic bacteria and indirectly preventing the human health from being affected due to food chain transfer action; 6. the selected polyvalent phage is derived from the soil and finally returned to the soil without any modification, and thus is environmentally friendly; 7. the ecological risk after application of the polyvalent phage therapy is evaluated to ensure the ecological functional diversity and stability of microorganisms.

### Advantageous Effect

Aiming at a method for targetedly inactivating compound high-abundance resistance pathogenic bacteria in a soil-vegetable system by polyvalent phage therapy, the present invention provides a rapid remediation technology. The present invention has the following main advantages: 1. resistance pathogenic bacteria in contaminated soil can be subjected to targeted inactivation, and the abundance of related resistance genes can be simultaneously reduced; 2. the phage therapy is low in preparation cost, convenient to storage and transport, simple and convenient to use and operate, accurate in inactivation, high in broad spectrum, capable of preventing “rebound” after remediation, and easy to popularize; 3. the phage is derived from the soil and returned to the soil, has a positive promotion effect on the ecological functional diversity and stability of soil microorganisms, and is environmentally friendly. The method has broad application prospects for remediation of compound high-concentration antibiotic resistance pathogenic bacterium and resistance gene contaminated soil around a large number of livestock and poultry farms in China.

### BRIEF DESCRIPTION OF THE DRAWINGS

FIG. 1 is a transmission electron micrograph of an exclusive phage φYSZKA with Klebsiella pneumoniae as host bacteria;

FIG. 2 is a transmission electron micrograph of a polyvalent phage φYSZPK capable of simultaneously attacking Klebsiella pneumoniae and Pseudomonas aeruginosa;

FIG. 3 is a transmission electron micrograph of an exclusive phage φYSZPA with Pseudomonas aeruginosa as host bacteria;

FIG. 4 is a verification diagram of an inactivation effect on compound pathogenic bacteria in a contaminated soil-vegetable system of a manure accumulation place of the Hengliang dairy farm in Nanjing, Jiangsu Province, when lettuces are planted on the contaminated soil, by using the technical solution of the present invention;

FIG. 5 is a verification diagram of an inactivation effect on compound pathogenic bacteria in a contaminated soil-vegetable system of the manure accumulation place of the Hengliang dairy farm in Nanjing, Jiangsu Province, when carrots are planted on the contaminated soil, by using the technical solution of the present invention;

FIG. 6 is a verification diagram of an inactivation effect on compound pathogenic bacteria in a contaminated soil-vegetable system of the manure accumulation place of the Hengliang dairy farm in Nanjing, Jiangsu Province, when pod peppers are planted on the contaminated soil, by using the technical solution of the present invention.

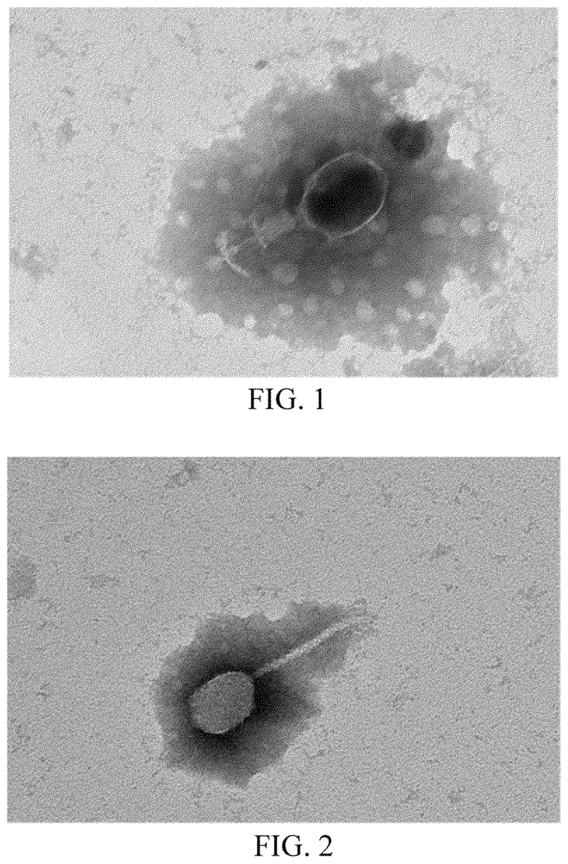

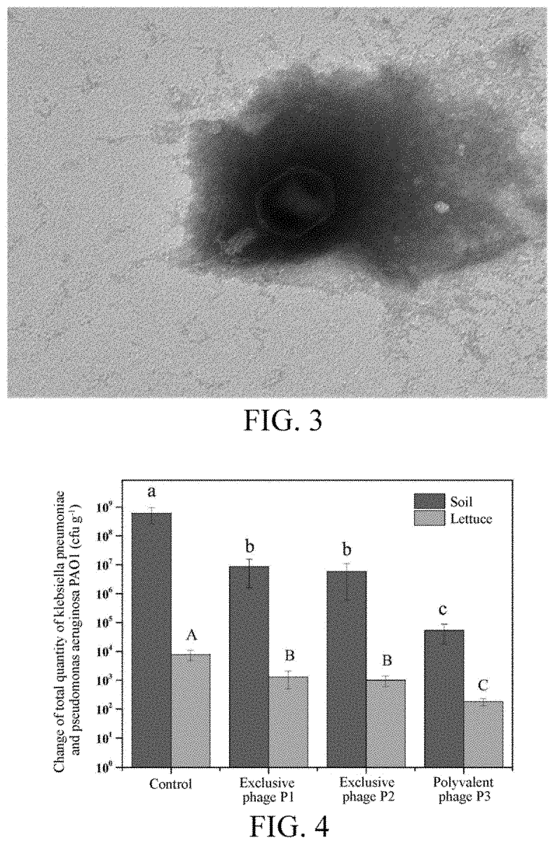

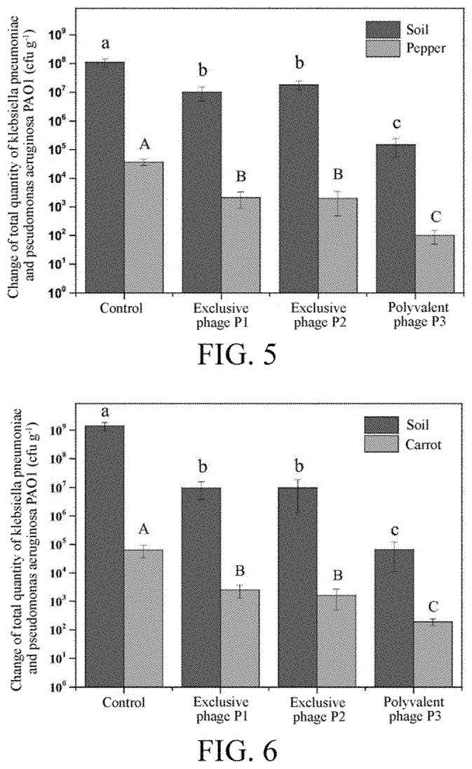

### DETAILED DESCRIPTION OF THE INVENTION

The following specific embodiments do not limit the technical solution of the present invention in any form. Any technical solution obtained by means of equivalent replacement or equivalent transformation falls within the protection scope of the present invention.

The phage φYSZKA is a previously deposited phage exclusively “preying on” Klebsiella pneumoniae, and the Accession No. of the phage φYSZKA is CCTCC M 2018513. The phage φYSZKA has a clear polyhedral head, and has a head length of about 95 nm, a transverse diameter of about 70 nm, and a tail length of about 110 nm. The phage φYSZKA has clear, transparent and round phage plaques with neat edges without halo, and with a diameter of about 1-2 mm. According to the ninth report of the International Committee on Taxonomy of Viruses (ICTV), the phage φYSZKA belongs to the Siphoviridae Bacteriophage.

The phage φYSZPK is a previously deposited polyvalent phage capable of simultaneously attacking Klebsiella pneumoniae and Pseudomonas aeruginosa PAO1, and the Accession No. of the phage φYSZPK is CCTCC M 2018514. The phage φYSZPK has a regular polyhedral head and a shrunk long tail, and has a head long diameter of about 70 nm, a transverse diameter of about 60 nm, and a tail length of about 180 nm. The phage φYSZPK has clear, transparent and round phage plaques with neat edges, without halo, and with a diameter of about 1-2 mm. According to the ninth report of the International Committee on Taxonomy of Viruses, the phage φYSZPK belongs to the Siphoviridae Bacteriophage.

The phage φYSZPA is a previously deposited phage exclusively “preying on” Pseudomonas aeruginosa PAO1, and the Accession No. of the phage φYSZPA is CCTCC M 2018515. The phage φYSZPA has a three-dimensional head in a regular shape and a long tail, and has a head long diameter of about 100 nm, a transverse diameter of about 70 nm, and a tail length of about 120 nm. The phage φYSZPA has phage plaques which are transparent in the middle, have no halo around, and have a diameter of about 2-3 mm. According to the ninth report of the International Committee on Taxonomy of Viruses, the phage φYSZPA belongs to the Siphoviridae Bacteriophage.

The resistance gene tetW refers to a resistance gene carrying related tetracycline on plasmids in Klebsiella pneumoniae cells.

The resistance gene ampC refers to a resistance gene carrying related chloramphenicol on plasmids in Pseudomonas aeruginosa PAO1 cells.

The potting soil is obtained by adding the same abundance of pathogenic bacteria (Klebsiella pneumoniae and Pseudomonas aeruginosa PAO1) to collected raw soil.

### Example 1

Test soil samples were collected from contaminated soil around a manure accumulation pool of the Hengliang dairy farm in Nanjing, Jiangsu Province. Basic physical and chemical properties of the soil were as follows: sand grain: 23.8%, soil grain: 45.4%, clay grain: 31.8%, pH: 7.7, total nitrogen: 1.7 g·kg⁻¹, water-soluble nitrogen: 1.7 g·kg⁻¹, total phosphorus: 1.3 g·kg⁻¹, total potassium: 17.5 g·kg⁻¹, and CEC: 19.4 cmol·kg⁻¹.

5 g of fresh soil samples were taken and added to 50 mL of sterile water, shake culture was performed for 5 h at 28° C. and 150 rpm, centrifugation was performed for 5 min at 10000 rpm, supernatant liquid was sterilized by a 0.22 μm filter membrane, 9 mL of filtrate and 1 mL of a suspension of Klebsiella pneumoniae grown to the logarithmic phase were taken and added to 40 mL of LB liquid culture medium, calcium chloride solids were added until the final concentration of the solution was 1 mmol·L⁻¹, shake culture was performed for 12 h at 30° C. and 150 rpm, the obtained culture solution was centrifuged for 5 min at 10000 rpm, and then, the centrifuged culture solution was sterilized by a 0.22 μm filter membrane to obtain a phage stock solution; phages were screened and purified by using a double-layer flat plate method, 100 μL of filtrate and 100 μL of Klebsiella pneumoniae suspension were taken and mixed uniformly, the mixture was allowed to stand for 15 min at room temperature, the mixture was added to 3 mL of 0.7% LB agar culture medium and horizontally poured on an LB solid flat plate after uniform mixing, culture was performed for 10-12 h at 30° C., phage plaques were observed, after the phage plaques occurred, a single phage plaque with clear and transparent edges was taken and put in LB liquid containing host bacteria, and culture was performed for 8 h at 30° C. and 250 rpm; and centrifugation was performed for 5 min at 10000 rpm, the centrifuged product was sterilized by a 0.22 μm filter membrane, the filtrate was preserved in an SM buffer solution, and the solution was refrigerated in a refrigerator at 4° C.

Based on the exclusive phage φYSZKA obtained by taking Klebsiella pneumoniae as host bacteria, the expression process of accelerating the broad host spectrum of the phage was carried out: 600 μL of preserved phage stock solution was taken and added to 99 mL of LB liquid culture medium together with 200 μL of Klebsiella pneumoniae and 200 mL of PAO1 mixed suspension, then, a calcium chloride solid was added until the final concentration was 1 mmol·L⁻¹, shake culture was performed for 96 h at 37° C. and 150 rpm, samples were taken every 8 h, the phage obtained by centrifugal filtration and PAO1 were poured into a double-layer flat plate to be verified, phage plaques were observed, if phage plaques occurred, it was proved that the directed evolution was successful, a polyvalent phage φYSZKP was obtained, a single clear and transparent phage plaque was selected and enriched, and mixed with 50% glycerol in a ratio of 1:1, and the mixture was preserved at low temperature of −80° C. for later use.

Two exclusive phages were obtained based on the above operation, and respectively were a Klebsiella pneumoniae exclusive phage φYSZKA and a Pseudomonas aeruginosa PAO1 phage φYSZPA, and a polyvalent phage φYSZKP capable of simultaneously attacking Klebsiella pneumoniae and Pseudomonas aeruginosa.

### Example 2

Test potting soil was collected from the contaminated soil around the manure accumulation pool of the Hengliang dairy farm in Nanjing, Jiangsu Province. Planting vegetables were Italian year-round bolting-resistant lettuces (Lactuca sativa L), and were derived from Hebei Jinfa Seed Industry Co., Ltd. Basic physical and chemical properties of the soil were as follows: sand grain: 23.8%, soil grain: 45.4%, clay grain: 31.8%, pH: 7.7, total nitrogen: 1.7 g·kg⁻¹, water-soluble nitrogen: 1.7 g·kg⁻¹, total phosphorus: 1.3 g·kg⁻¹, total potassium: 17.5 g·kg⁻¹, and CEC: 19.4 cmol·kg⁻¹.

Four groups of treatment were set in experiments: (1) control group (CK): 3 lettuces were planted per pot (0.5-1 cm of soil was covered on seeds, and the room temperature was 18±2° C.); (2) phage φYSZKA treatment (P1): 100 mL of exclusive phage φYSZKA with a concentration of 10⁶ pfu·mL⁻¹ was inoculated on the basis of the control group; (3) phage φYSZPA treatment (P2): 100 mL of exclusive phage φYSZPA with a concentration of 10⁶ pfu·mL⁻¹ was inoculated on the basis of the control group; (4) polyvalent phage φYSZKP treatment (P3): 100 mL of polyvalent phage φYSZKP with a concentration of 10⁶ pfu·mL⁻¹ was inoculated on the basis of the control group. The soil and lettuces were sampled on the site after the 60th day of lettuce growth, the measured background contamination concentrations of Klebsiella pneumoniae and Pseudomonas aeruginosa PAO1 in the contaminated soil of the control group were respectively 2.8×10⁷ cfu·g⁻¹ and 7.4×10⁷ cfu·g⁻¹, and the background contamination abundances of tetracycline resistance gene tetW and chloramphenicol resistance gene ampC were respectively 1.2×10⁸ copies·g⁻¹ and 1.4×10⁹ copies·g⁻¹. In the treatment of inoculating the phages φYSZKA, φYSZPA, and φYSZKP, the quantity of Klebsiella pneumoniae was respectively reduced to 1.3×10⁵ cfu·g⁻¹, 5.6×10⁶ cfu·g⁻¹, and 1.5×10⁵ cfu·g⁻¹, the abundance of the resistance gene tetW was respectively reduced to 8.3×10⁵ copies·g⁻¹, 5.7×10⁷ copies·g⁻¹, and 7.3×10⁵ copies·g⁻¹, the quantity of Pseudomonas aeruginosa PAO1 was respectively reduced to 8.5×10⁶ cfu·g⁻¹, 2.3×10⁵ cfu·g⁻¹, and 3.8×10⁵ cfu·g⁻¹, and the abundance of the resistance gene ampC was respectively reduced to 1.7×10⁸ copies·g⁻¹, 8.7×10⁶ copies·g¹, and 7.7×10⁶ copies·g¹. Compared with the control group (CK), in three groups of treatment of inoculating phages (P1, P2, and P3), the total quantity of Klebsiella pneumoniae and Pseudomonas aeruginosa was respectively reduced by 2.9, 3 and 4.4 orders of magnitude, and the total abundance of the resistance genes tetW and ampC was respectively reduced by 3.3, 3.9 and 4.7 orders of magnitude. The measured quantity of Klebsiella pneumoniae in lettuce leaves in four groups of treatment (CK, P1, P2, and P3) was respectively 3.2×10³ cfu·g¹, 1.8×10² cfu·g¹, 8.3×10² cfu·g⁻¹, and 4.2×10² cfu·g⁻¹, the abundance of the resistance gene tetW was respectively reduced to 1.7×10⁴ copies·g⁻¹, 8.2×10² copies·g⁻¹, 4.2×10³ copies·g⁻¹, and 2.2×10² copies·g⁻¹, PAO1 was respectively reduced to 3.8×10³ cfu·g⁻¹, 8.2×10² cfu·g⁻¹, 1.9×10² cfu·g⁻¹, and 1.4×10² cfu·g¹, and the abundance of the resistance gene ampC was respectively reduced to 7.7×10⁴ copies·g⁻¹, 4.5×10³ copies·g⁻¹, 7.5×10² copies·g⁻¹, and 2.8×10² copies·g⁻¹. Compared with the control group, the total quantity of Klebsiella pneumoniae and Pseudomonas aeruginosa in leaves was respectively reduced by 1.6, 1.7 and 2.1 orders of magnitude, and the total abundance of the resistance genes tetW and ampC in leaves was respectively reduced by 2.7, 2.8 and 3.5 orders of magnitude. The inactivation effect of the polyvalent phage φYSZKP for resistance pathogenic bacteria and resistance genes was significantly higher than that of the exclusive phages.

The analysis finds that ecological diversity indexes (AWCD indexes) of microorganisms in the soil environment under four groups of treatment (CK, P1, P2, and P3) were respectively 0.54±0.1, 0.50±0.2, 0.51±0.1, and 0.55±0.2, by P1 treatment and P2 treatment of inoculating the exclusive phages, the diversity of microorganisms in soil was reduced to a certain degree, and by inoculating the polyvalent phage P3, the functional diversity and stability of microorganisms in soil after remediation were significantly promoted (p<0.05), indicating that the remediation technology has a significant effect on remediation of the contamination of resistance bacteria.

### Example 3

Test potting soil was collected from the contaminated soil around the manure accumulation pool of the Hengliang dairy farm in Nanjing, Jiangsu Province. Planting vegetables were carrots (DaucusL.), and were derived from Beijing Zhongnong Tianteng Vegetable Seed Company. Basic physical and chemical properties of the soil were as follows: sand grain: 23.8%, soil grain: 45.4%, clay grain: 31.8%, pH: 7.7, total nitrogen: 1.7 g·kg⁻¹, water-soluble nitrogen: 1.7 g·kg⁻¹, total phosphorus: 1.3 g·kg⁻¹, total potassium: 17.5 g·kg⁻¹, and CEC: 19.4 cmol·kg⁻¹.

Four groups of treatment were set in experiments: (1) control group (CK): 3 carrots were planted per pot (0.5-1 cm of soil was covered on seeds, and the room temperature was 20±2° C.); (2) phage φYSZKA treatment (P1): 100 mL of exclusive phage φYSZKA with a concentration of 10⁶ pfu·mL⁻¹ was inoculated on the basis of the control group; (3) phage φYSZPA treatment (P2): 100 mL of exclusive phage φYSZPA with a concentration of 10⁶ pfu·mL⁻¹ was inoculated on the basis of the control group; (4) polyvalent phage φYSZKP treatment (P3): 100 mL of polyvalent phage φYSZKP with a concentration of 10⁶ pfu·mL⁻¹ was inoculated on the basis of the control group. The soil and carrots were sampled on the site after the 70th day of carrot growth, the measured background contamination concentrations of Klebsiella pneumoniae and Pseudomonas aeruginosa PAO1 in the contaminated soil of the control group were respectively 3.8×10⁷ cfu·g⁻¹ and 5.4×10⁷ cfu·g⁻¹, and the background contamination abundances of the tetracycline resistance gene tetW and the chloramphenicol resistance gene ampC were respectively 1.6×10⁸ copies·g⁻¹ and 2.3×10⁹ copies·g⁻¹. In P1, P2, and P3 treatment of inoculating the phages, the quantity of Klebsiella pneumoniae was respectively reduced to 1.6×10⁵ cfu·g⁻¹, 9.2×10⁶ cfu·g⁻¹, and 1.8×10⁵ cfu·g⁻¹, the abundance of the resistance gene tetW was respectively reduced to 8.1×10⁵ copies·g⁻¹, 4.7×10⁷ copies·g⁻¹, and 7.8×10⁵ copies·g⁻¹, the quantity of Pseudomonas aeruginosa PAO1 was respectively reduced to 9.5×10⁶ cfu·g⁻¹, 5.3×10⁵ cfu·g⁻¹, and 4.8×10⁵ cfu·g⁻¹, and the abundance of the resistance gene ampC was respectively reduced to 1.9×10⁸ copies·g⁻¹, 1.4×10⁶ copies·g⁻¹, and 7.8×10⁶ copies·g⁻¹. Compared with the control group (CK), in three groups of treatment (P1, P2, and P3), the total quantity of Klebsiella pneumoniae and Pseudomonas aeruginosa PAO1 was respectively reduced by 2.7, 2.8 and 4.2 orders of magnitude, and the total abundance of the resistance genes tetW and ampC was respectively reduced by 3.4, 3.6 and 4.8 orders of magnitude. The measured related quantity of Klebsiella pneumoniae in carrot root tubers in four groups of treatment (CK, P1, P2, and P3) was respectively 5.2×10⁴ cfu·g⁻¹, 2.8×10² cfu·g⁻¹, 1.3×10³ cfu·g⁻¹, and 3.2×10² cfu·g⁻¹, the abundance of the resistance gene tetW was respectively reduced to 2.6×10⁵ copies·g⁻¹, 1.8×10³ copies·g⁻¹, 8.3×10⁴ copies·g⁻¹, and 1.9×10³ copies·g⁻¹, PAO1 was respectively reduced to 8.2×10³ cfu·g⁻¹, 2.2×10³ cfu·g⁻¹, 2.3×10² cfu·g⁻¹, and 3.7×10² cfu·g⁻¹, and the abundance of the resistance gene ampC was respectively reduced to 1.9×10⁵ copies·g⁻¹, 4.2×10⁴ copies·g⁻¹, 4.8×10³ copies·g⁻¹, and 5.8×10³ copies·g⁻¹. Compared with the control group, the total quantity of Klebsiella pneumoniae and Pseudomonas aeruginosa PAO1 in root tubers was respectively reduced by 1.5, 1.9 and 2.2 orders of magnitude, and compared with the control group, the total abundance of the resistance genes tetW and ampC in root tubers was respectively reduced by 2.9, 2.2 and 3.7 orders of magnitude. The effect of the polyvalent phage was significantly better than that of the exclusive phages.

The analysis finds that the ecological diversity indexes (AWCD indexes) of microorganisms in the soil environment under four groups of treatment (CK, P1, P2, and P3) were respectively 0.68±0.2, 0.64±0.2, 0.65±0.1, and 0.70±0.2, by P1 treatment and P2 treatment of inoculating the exclusive phages, the diversity of microorganisms in soil was reduced to a certain degree, and by inoculating the polyvalent phage (P3), the functional diversity and stability of microorganisms in soil after remediation were significantly promoted to a certain degree (p<0.05), indicating that the remediation technology has a significant effect on remediation of the spread of resistance bacteria, and was also favorable for maintaining and improving the ecological functional diversity and stability of microorganisms in soil after remediation.

### Example 4

Test potting soil was collected from the contaminated soil around the manure accumulation pool of the Hengliang dairy farm in Nanjing, Jiangsu Province. Planting vegetables were Hongpin No. 1 pod peppers (Capsicum frutescens var), and were derived from Qianshu Baihua Seed Industry. Basic physical and chemical properties of the soil were as follows: sand grain: 23.8%, soil grain: 45.4%, clay grain: 31.8%, pH: 7.7, total nitrogen: 1.7 g·kg⁻¹, water-soluble nitrogen: 1.7 g·kg⁻¹, total phosphorus: 1.3 g·kg⁻¹, total potassium: 17.5 g·kg⁻¹, and CEC: 19.4 cmol·kg⁻¹.

Four groups of treatment were set in experiments: (1) control group (CK): three pod peppers were planted per pot (0.5-1 cm of soil was covered on seeds, and the room temperature was 25±2° C.); (2) phage φYSZKA treatment (P1): 100 mL of exclusive phage φYSZKA with a concentration of 10⁶ pfu·mL⁻¹ was inoculated on the basis of the control group; (3) phage φYSZPA treatment (P2): 100 mL of exclusive phage φYSZPA with a concentration of 10⁶ pfu·mL⁻¹ was inoculated on the basis of the control group; (4) polyvalent phage φYSZKP treatment (P3): 100 mL of polyvalent phage φYSZKP with a concentration of 10⁶ pfu·mL⁻¹ was inoculated on the basis of the control group. The soil and pod peppers were sampled on the site after the 70th day of pod pepper growth, the measured background contamination concentrations of Klebsiella pneumoniae and Pseudomonas aeruginosa PAO1 in the contaminated soil were respectively 6.2×10⁷ cfu·g⁻¹ and 5.5×10⁷ cfu·g⁻¹, and the background contamination abundances of the tetracycline resistance gene tetW and the chloramphenicol resistance gene ampC were respectively 3.3×10⁸ copies·g⁻¹ and 1.3×10⁹ copies·g⁻¹. In treatment of inoculating the phages φYSZKA, φYSZPA, and φYSZKP, the quantity of Klebsiella pneumoniae was respectively reduced to 3.7×10⁵ cfu·g⁻¹, 1.8×10⁷ cfu·g⁻¹, and 6.3×10⁵ cfu·g⁻¹, the abundance of the resistance gene tetW was respectively reduced to 9.5×10⁶ copies·g⁻¹, 9.2×10⁷ copies·g⁻¹, and 3.8×10⁶ copies·g⁻¹, the quantity of Pseudomonas aeruginosa PAO1 was respectively reduced to 3.8×10⁷ cfu·g⁻¹, 3.2×10⁴ cfu·g⁻¹, and 3.5×10⁵ cfu·g⁻¹, and the abundance of the resistance gene ampC was respectively reduced to 7.8×10⁷ copies·g⁻¹, 1.7×10⁶ copies·g⁻¹, and 6.5×10⁶ copies·g⁻¹. Compared with the control group (CK), in three groups of treatment (P1, P2, and P3) of inoculating the phages, the total quantity of Klebsiella pneumoniae and Pseudomonas aeruginosa PAO1 was respectively reduced by 2.3, 2.6 and 4.1 orders of magnitude, and the total abundance of the resistance genes tetW and ampC was respectively reduced by 2.7, 3.3 and 4.2 orders of magnitude. The measured quantity of Klebsiella pneumoniae in pod pepper fruits in four groups of treatment (CK, P1, P2, and P3) was respectively reduced to 3.2×10³ cfu·g⁻¹, 1.8×10² cfu·g⁻¹, 8.3×10² cfu·g⁻¹, and 4.2×10² cfu·g⁻¹, the abundance of the resistance gene tetW was respectively reduced to 1.6×10⁴ copies·g⁻¹, 8.3×10² copies·g⁻¹, 4.2×10³ copies·g⁻¹, and 9.2×10² copies·g⁻¹, Pseudomonas aeruginosa PAO1 was respectively 3.8×10³ cfu·g⁻¹, 8.2×10² cfu·g⁻¹, 1.9×10² cfu·g⁻¹, and 1.4×10² cfu·g⁻¹, and the abundance of the resistance gene ampC was respectively reduced to 2.6×10⁴ copies·g⁻¹, 5.8×10³ copies·g⁻¹, 3.8×10² copies·g⁻¹, and 2.8×10² copies·g⁻¹. Compared with the control group, the total abundance of Klebsiella pneumoniae and Pseudomonas aeruginosa PAO1 was respectively reduced by 1.6, 1.7 and 2.1 orders of magnitude, and compared with the control group, the total abundance of the resistance genes tetW and ampC in fruits was respectively reduced by 2.1, 2.7 and 3.3 orders of magnitude. The effect of the polyvalent phage was significantly better than that of the exclusive phages.

The analysis finds that the ecological diversity indexes (AWCD indexes) of microorganisms in the soil environment under four groups of treatment (CK, P1, P2, and P3) were respectively 0.74±0.1, 0.70±0.2, 0.71±0.1, and 0.75±0.2, by P1 treatment and P2 treatment of inoculating the exclusive phages, the diversity of microorganisms in soil was reduced to a certain degree, and by inoculating the polyvalent phage (P3), the functional diversity and stability of microorganisms in soil after remediation were significantly promoted to a certain degree (p<0.05), thereby indicating that the remediation technology has a significant effect on remediation of the spread of resistance bacteria, and was also favorable for maintaining and improving the ecological functional diversity and stability of microorganisms in soil after remediation.

The technology for simultaneous inactivation of multiple resistance pathogenic bacteria in a soil-vegetable system by polyvalent phage therapy has the advantages of high broad spectrum, low ecological risk and environmental friendliness, and is a compound pathogenic bacterium contaminated soil remediation technology with good application prospects.

What is claimed is:

1. A phage composition, comprising three phages, wherein the phages all have been deposited at the China Center for Type Culture Collection on Aug. 1, 2018, and the phages are a phage φYSZKA under Accession No. CCTCC M 2018513 whose taxonomic designation is Klebsiella phage φYSZKA, a phage φYSZKP under Accession No. CCTCC M 2018514 whose taxonomic designation is Klebsiella and Pseudomonas aeruginosa phage φYSZKP, and a phage φYSZPA under Accession No. CCTCC M 2018515 whose taxonomic designation is Pseudomonas aeruginosa phage φYSZPA, respectively.

2. Use of a phage composition according to claim 1 for targetedly inactivating antibiotic resistance pathogenic bacteria in a soil-vegetable system.

3. Use of a phage composition according to claim 1 for preparing a product for targetedly inactivating antibiotic resistance pathogenic bacteria in a soil-vegetable system.
